(19) United States
(12) Patent Application Publication
(10) Pub. No.: US 2014/0322358 A1
(43) Pub. Date: Oct. 30, 2014

(54) Compositions and methods for amelioration of chronic renal failure and related symptoms in domestic animals

(71) Applicant: Jeffrey Alan McKinney, Lafayette, CA (US)

(72) Inventor: Jeffrey Alan McKinney, Lafayette, CA (US)

(21) Appl. No.: 14/120,103

(22) Filed: Apr. 24, 2014

Related U.S. Application Data
(60) Provisional application No. 61/854,509, filed on Apr. 25, 2013.

(51) Int. Cl. A61K 33/14; A61K 36/45; A61K 36/73; A61K 36/752; A61K 36/21; A61K 36/87; A61K 36/31; A61K 36/23; A61K 36/06; A61K 36/81; A61K 36/18

(52) U.S. Cl. CPC A61K 33/14; A61K 36/81; A61K 36/45; A61K 36/73; A61K 36/752; A61K 36/18; A61K 36/87; A61K 36/31; A61K 36/23; A61K 36/06; A61K 36/21
USPC 424/677; 424/725; 424/732; 424/765; 424/736; 424/766; 424/755; 424/777

(57) ABSTRACT

The present invention is generally directed to compositions and methods for amelioration chronic renal failure and related symptoms in domestic animals. In one case, the present invention is directed to a composition for the treatment of chronic renal failure in a domestic animal or symptoms related to chronic renal failure. The composition comprises a Fruit- and Vegetable-Based Additive. In another case, the present invention is directed to a composition for the treatment of chronic renal failure in a domestic animal or symptoms related to chronic renal failure. The composition comprises a Cesium and/or Rubidium salt. In another case, the present invention is directed to a composition for the treatment of chronic renal failure in a domestic animal or symptoms related to chronic renal failure. The composition comprises a Dehydrated Domestic Animal Food and a Phosphate Binder.

### FIELD OF THE INVENTION

The present invention is generally directed to compositions and methods for amelioration chronic renal failure and related symptoms in domestic animals.

### BACKGROUND OF THE INVENTION

There have been reports regarding the treatment of kidney ailments such as chronic renal failure. For example, U.S. Pat. No. 5,039,709, issued Aug. 13, 1991 to BASF Aktiengesellschaft, is allegedly directed to the “[u]se of anipamil and its sales with physiologically tolerated acids for the treatment of chronic renal failure . . . ” Abstract. U.S. Pat. No. 5,591,736, issued Jan. 7, 1997 to The Johns Hopkins University, includes the following discussion: “The progression of chronic renal failure in humans may be retarded by administration of dehydroepiandrosterone (DHEA) in effective amounts. The administration of DHEA is preferably oral at a dose of about 400 to 1600 mg/day. Patients suffering from severe chronic renal failure are also preferably maintained on a protein-restricted diet during DHEA administration.” Abstract.

U.S. Pat. No. 5,723,441, issued Mar. 3, 1998 to Celtrix Pharmaceuticals, Inc., includes the following discussion: “Treatment with IGF/IGFBP-3 complex increases renal tubular mass and potentiates and/or stimulates kidney function in subjects suffering from acute and chronic renal failure or insufficiency resulting from such disorders as glomerulonephritis, glomerulosclerosis, interstitial nephritis, acute tubular necrosis due to ischemia and drug-induced toxicity, diabetic and autoimmune nephropathies and renal dysfunction due to acute and chronic rejection episodes in post-translation patients.” Abstract. U.S. Pat. No. 6,498,142, issued Dec. 24, 2002 to Curis, Inc., includes the following discussion: “The present invention provides methods for the treatment, and pharmaceuticals for use in the treatment, of mammalian subjects at risk chronic renal failure, or at risk of a need for renal replacement therapy. The methods involve the administration of certain morphogens, inducers of those morphogens, or agonists of the corresponding morphogen receptors, or implantation of renal cells induced with those morphogens. The morphogens useful in the invention include osteogenic protein-1 (OP-1) and other members of the OP-1 subfamily of the TGF-β superfamily of growth factors.” Abstract.

U.S. Pat. No. 6,610,719, issued Aug. 26, 2003 to Pfizer, Inc., includes the following discussion: “This invention is directed to methods and compositions of treating acute or chronic renal failure or dysfunction, or conditions caused thereby, comprising administering prostaglandin agonists, which are EP₄ receptor selective prostaglandin agonists.” Abstract. U.S. Pat. No. 7,612,046, issued Nov. 3, 2009 to Rothenpieler et al., includes the following discussion: “The present invention relates to a cellular therapy of renal failure, i.e., preventing, delaying, and treating acute/chronic renal failure via the use of an effective dose of a substance capable of inducing and/or enhancing Pax2 expression in a mammal for the preparation of a pharmaceutical composition for treating, preventing or delaying a renal dysfunction/failure in a mammal. Additionally, the present invention provides for a method for converting mesenchymal tissue into an epithelial tissue comprising the administration of an effective amount of a substance capable of inducing and/or enhancing Pax2 expression in mesenchymal tissue and for a method for the regeneration of renal stem cells comprising the administration of an effective amount of a substance capable of inducing and/or enhancing Pax2 expression.” Abstract.

Despite various reports, there is still a need for new compositions and methods for amelioration of chronic renal failure and related symptoms in domestic animals.

### SUMMARY OF THE INVENTION

In one case, the present invention is directed to a composition for the treatment of chronic renal failure in a domestic animal or symptoms related to chronic renal failure. The composition comprises a Fruit- and Vegetable-Based Additive.

In another case, the present invention is directed to a composition for the treatment of chronic renal failure in a domestic animal or symptoms related to chronic renal failure. The composition comprises a Cesium and/or Rubidium salt.

In another case, the present invention is directed to a composition for the treatment of chronic renal failure in a domestic animal or symptoms related to chronic renal failure. The composition comprises a Dehydrated Domestic Animal Food and a Phosphate Binder.

### DETAILED DESCRIPTION OF THE INVENTION

The present invention is generally directed to compositions and methods for amelioration chronic renal failure and related symptoms in domestic animals.

“Cesium Salt” refers to a compound that includes a positively charged cesium ion and a negatively charged counterion. Nonlimiting examples of such compounds include: cesium arginate, cesium ascorbate, cesium bicarbonate, cesium caprylate, cesium chloride, cesium cysteinate, cesium citrate, cesium fumarate, cesium humate, cesium fulvate, cesium methionate, cesium glutamate, cesium gluconate, cesium glycinate, cesium aspartate, cesium lysinate, cesium succinate, cesium carbonate, cesium lactate, cesium malate, cesium tartrate, cesium sulfate, cesium phosphate, cesium nitrate, cesium fluoride, cesium bromide, cesium iodide, cesium orotate, and mixtures thereof.

“Dehydrated Domestic Animal Food” refers to food materials suitable for domestic animal feeding that have been dehydrated and which can be reconstituted upon mixing with one or more liquids. The food can include a single ingredient (e.g., chicken or fish) or multiple ingredients. The ingredients can be raw or cooked. Nonlimiting examples of ingredients include: chicken, fish, beef, lamb, venison, eggs, potatoes, sweet potatoes, flaxseed, pumpkin, spinach, cranberries, and mixtures thereof.

“Domestic Animal” refers to any of various animals that have been tamed and made fit for a human environment. Nonlimiting examples of Domestic Animals include: cats, dogs and horses.

“Fruit-Based Additive” refers to a fruit extract or concentrate. The extract or concentrate includes material from a single or from multiple fruits. One method of making a Fruit-Based Additive concentrate is freeze drying. Nonlimiting examples of fruits that are used to make the additive include dried forms of the following: tomato, cranberry, apple, orange, pomegranate, grape, strawberry, and mixtures thereof.

“Fruit- and Vegetable-Based Additive” refers to a fruit and vegetable extract or concentrate. The extract or concentrate includes material from a single fruit and vegetable or from multiple fruits and vegetables. One method of making a Fruit- and Vegetable-Based Additive concentrate is freeze drying. Nonlimiting examples of fruits and vegetables that are used to make the additive include dried forms of the following: tomato, cranberry, apple, orange, pomegranate, grape, strawberry, broccoli, carrot, shitake mushrooms, spinach, beet, and mixtures thereof. In one case, the additive includes the following fruits and vegetables: tomato, broccoli, carrot, shitake mushrooms, cranberry, apple, and orange. In another case, the additive includes the following fruits and vegetables: cranberry, pomegranate, orange, grape, strawberry, and shitake mushrooms. In still another case, the additive includes the following fruits and vegetables: spinach, broccoli, carrot, tomato, beet, and shitake mushrooms.

“Phosphate Binder” refers to a group of compounds used to reduce the absorption of phosphate by a domestic animal when taken with meals and snacks. Nonlimiting examples of phosphate binders include: aluminum hydroxide, aluminum magnesium hydroxide, calcium acetate, calcium carbonate, chitosan, ferric citrate, lanthanum carbonate, magnesium carbonate, sevelamer, and mixtures thereof.

“Rubidium Salt” refers to a compound that includes a positively charged rubidium ion and a negatively charged counterion. Nonlimiting examples of such compounds include: rubidium arginate, rubidium ascorbate, rubidium bicarbonate, rubidium caprylate, rubidium chloride, rubidium cysteinate, rubidium citrate, rubidium fumarate, rubidium humate, rubidium fulvate, rubidium methionate, rubidium glutamate, rubidium gluconate, rubidium glycinate, rubidium aspartate, rubidium lysinate, rubidium succinate, rubidium carbonate, rubidium lactate, rubidium malate, rubidium tartrate, rubidium sulfate, rubidium phosphate, rubidium nitrate, rubidium fluoride, rubidium bromide, rubidium iodide, rubidium orotate, and mixtures thereof.

“Symptoms of Chronic Renal Failure” refers to the following nonlimiting list: appetite loss/decrease, weight loss, vomiting or diarrhea, lethargy or depression, dehydration, change in water consumption, pain in the kidney area, litter box aversion, mouth ulcers, bad breath, constipation, bloody or cloudy urine, urinating in abnormal places or pain when urinating, stumbling.

“Vegetable-Based Additive” refers to a vegetable extract or concentrate. The extract includes material from a single vegetable or from multiple vegetables. One method of making a Vegetable-Based Additive concentrate is freeze drying. Nonlimiting examples of vegetables that are used to make the additive include dried forms of the following: broccoli, carrot, shitake mushrooms, spinach, beet, and mixtures thereof.

In one case, compositions according to the present invention comprise a Cesium Salt and/or a Rubidium Salt.

Nonlimiting examples of such compositions include: a composition comprising cesium chloride; a composition comprising cesium bicarbonate; a composition comprising rubidium chloride.

In another case, compositions according to the present invention comprise a Fruit-Based, Vegetable-Based, or a Fruit- and Vegetable-Based Additive.

Nonlimiting examples of such compositions include: a Fruit- and Vegetable-Based Additive comprising tomato, broccoli, carrot, shitake mushrooms, cranberry, apple, and orange; a Fruit- and Vegetable-Based Additive comprising cranberry, pomegranate, orange, grape, strawberry, and shitake mushrooms; a Fruit- and Vegetable-Based Additive comprising spinach, broccoli, carrot, tomato, beet, and shitake mushrooms.

In another case, compositions according to the present invention comprise Dehydrated Domestic Animal Food and a Phosphate Binder.

Nonlimiting examples of such compositions include: a composition comprising dehydrated chicken and chitosan; a composition comprising dehydrated fish and chitosan; a composition comprising dehydrated beef and chitosan; a composition comprising dehydrated chicken and calcium carbonate; a composition comprising dehydrated fish and calcium carbonate; a composition comprising dehydrated beef and calcium carbonate; a composition comprising dehydrated chicken, chitosan and calcium carbonate; a composition comprising dehydrated fish, chitosan and calcium carbonate; a composition comprising dehydrated beef, chitosan and calcium carbonate.

In another case, compositions according to the present invention comprise Dehydrated Domestic Animal Food and a Cesium Salt and/or a Rubidium Salt.

Nonlimiting examples of such compositions include: a composition comprising dehydrated chicken and cesium chloride; a composition comprising dehydrated chicken and cesium bicarbonate; a composition comprising dehydrated fish and cesium chloride; a composition comprising dehydrated fish and cesium bicarbonate.

In another case, compositions according to the present invention comprise Dehydrated Domestic Animal Food and a Fruit-Based, Vegetable-Based, or a Fruit- and Vegetable-Based Additive.

Nonlimiting examples of such compositions include: a composition comprising dehydrated chicken and a Fruit- and Vegetable-Based Additive comprising tomato, broccoli, carrot, shitake mushrooms, cranberry, apple, and orange; a composition comprising dehydrated fish and a Fruit- and Vegetable-Based Additive comprising tomato, broccoli, carrot, shitake mushrooms, cranberry, apple, and orange; a composition comprising dehydrated chicken and a Fruit- and Vegetable-Based Additive comprising cranberry, pomegranate, orange, grape, strawberry, and shitake mushrooms; a composition comprising dehydrated fish and a Fruit- and Vegetable-Based Additive comprising spinach, broccoli, carrot, tomato, beet, and shitake mushrooms; a composition comprising dehydrated chicken and a Fruit- and Vegetable-Based Additive comprising cranberry, pomegranate, orange, grape, strawberry, and shitake mushrooms; a composition comprising dehydrated fish and a Fruit- and Vegetable-Based Additive comprising spinach, broccoli, carrot, tomato, beet, and shitake mushrooms.

In another case compositions according to the present invention comprise Dehydrated Domestic Animal Food, a Phosphate Binder, and a Cesium Salt and/or a Rubidium Salt.

Nonlimiting examples of such compositions include: a composition comprising dehydrated chicken, chitosan, and cesium chloride; a composition comprising dehydrated chicken, calcium carbonate, and cesium chloride; a composition comprising dehydrated chicken, chitosan, and cesium bicarbonate; a composition comprising dehydrated chicken, calcium carbonate, and cesium bicarbonate; a composition comprising dehydrated fish, chitosan, and cesium chloride; a composition comprising dehydrated fish, calcium carbonate, and cesium chloride; a composition comprising dehydrated fish, chitosan, and cesium bicarbonate; a composition comprising dehydrated fish, calcium carbonate, and cesium bicarbonate.

In another case compositions according to the present invention comprise Dehydrated Domestic Animal Food, a Phosphate Binder, and a Fruit-Based, Vegetable-Based, or a Fruit- and Vegetable-Based Additive.

Nonlimiting examples of such compositions include: a composition comprising dehydrated chicken, chitosan, and a Fruit- and Vegetable-Based Additive comprising tomato, broccoli, carrot, shitake mushrooms, cranberry, apple, and orange; a composition comprising dehydrated chicken, chitosan, and a Fruit- and Vegetable-Based Additive comprising cranberry, pomegranate, orange, grape, strawberry, and shitake mushrooms; a composition comprising dehydrated chicken, chitosan, and a Fruit- and Vegetable-Based Additive comprising spinach, broccoli, carrot, tomato, beet, and shitake mushrooms; a composition comprising dehydrated fish, chitosan, and a Fruit- and Vegetable-Based Additive comprising tomato, broccoli, carrot, shitake mushrooms, cranberry, apple, and orange; a composition comprising dehydrated fish, chitosan, and a Fruit- and Vegetable-Based Additive comprising cranberry, pomegranate, orange, grape, strawberry, and shitake mushrooms; a composition comprising dehydrated fish, chitosan, and a Fruit- and Vegetable-Based Additive comprising spinach, broccoli, carrot, tomato, beet, and shitake mushrooms; a composition comprising dehydrated chicken, calcium carbonate, and a Fruit- and Vegetable-Based Additive comprising tomato, broccoli, carrot, shitake mushrooms, cranberry, apple, and orange; a composition comprising dehydrated chicken, calcium carbonate, and a Fruit- and Vegetable-Based Additive comprising cranberry, pomegranate, orange, grape, strawberry, and shitake mushrooms; a composition comprising dehydrated chicken, calcium carbonate, and a Fruit- and Vegetable-Based Additive comprising spinach, broccoli, carrot, tomato, beet, and shitake mushrooms; a composition comprising dehydrated fish, calcium carbonate, and a Fruit- and Vegetable-Based Additive comprising tomato, broccoli, carrot, shitake mushrooms, cranberry, apple, and orange; a composition comprising dehydrated fish, calcium carbonate, and a Fruit- and Vegetable-Based Additive comprising cranberry, pomegranate, orange, grape, strawberry, and shitake mushrooms; a composition comprising dehydrated fish, calcium carbonate, and a Fruit- and Vegetable-Based Additive comprising spinach, broccoli, carrot, tomato, beet, and shitake mushrooms.

In another case, compositions according to the present invention comprise Dehydrated Domestic Animal Food, a Cesium Salt and/or a Rubidium Salt, and a Fruit-Based, Vegetable-Based, or a Fruit- and Vegetable-Based Additive.

Nonlimiting examples of such compositions include: a composition comprising dehydrated chicken, cesium chloride, and a Fruit- and Vegetable-Based Additive comprising tomato, broccoli, carrot, shitake mushrooms, cranberry, apple, and orange; a composition comprising dehydrated chicken, cesium chloride, and a Fruit- and Vegetable-Based Additive comprising cranberry, pomegranate, orange, grape, strawberry, and shitake mushrooms; a composition comprising dehydrated chicken, cesium chloride, and a Fruit- and Vegetable-Based Additive comprising spinach, broccoli, carrot, tomato, beet, and shitake mushrooms; a composition comprising dehydrated fish, cesium chloride, and a Fruit- and Vegetable-Based Additive comprising tomato, broccoli, carrot, shitake mushrooms, cranberry, apple, and orange; a composition comprising dehydrated fish, cesium chloride, and a Fruit- and Vegetable-Based Additive comprising cranberry, pomegranate, orange, grape, strawberry, and shitake mushrooms; a composition comprising dehydrated fish, cesium chloride, and a Fruit- and Vegetable-Based Additive comprising spinach, broccoli, carrot, tomato, beet, and shitake mushrooms; a composition comprising dehydrated chicken, cesium bicarbonate, and a Fruit- and Vegetable-Based Additive comprising tomato, broccoli, carrot, shitake mushrooms, cranberry, apple, and orange; a composition comprising dehydrated chicken, cesium bicarbonate, and a Fruit- and Vegetable-Based Additive comprising cranberry, pomegranate, orange, grape, strawberry, and shitake mushrooms; a composition comprising dehydrated chicken, cesium bicarbonate, and a Fruit- and Vegetable-Based Additive comprising spinach, broccoli, carrot, tomato, beet, and shitake mushrooms; a composition comprising dehydrated fish, cesium bicarbonate, and a Fruit- and Vegetable-Based Additive comprising tomato, broccoli, carrot, shitake mushrooms, cranberry, apple, and orange; a composition comprising dehydrated fish, cesium bicarbonate, and a Fruit- and Vegetable-Based Additive comprising cranberry, pomegranate, orange, grape, strawberry, and shitake mushrooms; a composition comprising dehydrated fish, cesium bicarbonate, and a Fruit- and Vegetable-Based Additive comprising spinach, broccoli, carrot, tomato, beet, and shitake mushrooms.

In another case, compositions according to the present invention comprise Dehydrated Domestic Animal Food, a Phosphate Binder, a Cesium Salt and/or a Rubidium Salt, and a Fruit-Based, Vegetable-Based, or a Fruit- and Vegetable-Based Additive.

Nonlimiting examples of such compositions include: a composition comprising dehydrated chicken, chitosan, cesium chloride, and a Fruit- and Vegetable-Based Additive comprising tomato, broccoli, carrot, shitake mushrooms, cranberry, apple, and orange; a composition comprising dehydrated chicken, chitosan, cesium chloride, and a Fruit- and Vegetable-Based Additive comprising cranberry, pomegranate, orange, grape, strawberry, and shitake mushrooms; a composition comprising dehydrated chicken, chitosan, cesium chloride, and a Fruit- and Vegetable-Based Additive comprising spinach, broccoli, carrot, tomato, beet, and shitake mushrooms; a composition comprising dehydrated fish, chitosan, cesium chloride, and a Fruit- and Vegetable-Based Additive comprising tomato, broccoli, carrot, shitake mushrooms, cranberry, apple, and orange; a composition comprising dehydrated fish, chitosan, cesium chloride, and a Fruit- and Vegetable-Based Additive comprising cranberry, pomegranate, orange, grape, strawberry, and shitake mushrooms; a composition comprising dehydrated fish, chitosan, cesium chloride, and a Fruit- and Vegetable-Based Additive comprising spinach, broccoli, carrot, tomato, beet, and shitake mushrooms; a composition comprising dehydrated chicken, chitosan, cesium bicarbonate, and a Fruit- and Vegetable-Based Additive comprising tomato, broccoli, carrot, shitake mushrooms, cranberry, apple, and orange; a composition comprising dehydrated chicken, chitosan, cesium bicarbonate, and a Fruit- and Vegetable-Based Additive comprising cranberry, pomegranate, orange, grape, strawberry, and shitake mushrooms; a composition comprising dehydrated chicken, chitosan, cesium bicarbonate, and a Fruit- and Vegetable-Based Additive comprising spinach, broccoli, carrot, tomato, beet, and shitake mushrooms; a composition comprising dehydrated fish, chitosan, cesium bicarbonate, and a Fruit- and Vegetable-Based Additive comprising tomato, broccoli, carrot, shitake mushrooms, cranberry, apple, and orange; a composition comprising dehydrated fish, chitosan, cesium bicarbonate, and a Fruit- and Vegetable-Based Additive comprising cranberry, pomegranate, orange, grape, strawberry, and shitake mushrooms; a composition comprising dehydrated fish, chitosan, cesium bicarbonate, and a Fruit- and Vegetable-Based Additive comprising spinach, broccoli, carrot, tomato, beet, and shitake mushrooms; a composition comprising dehydrated chicken, calcium carbonate, cesium chloride, and a Fruit- and Vegetable-Based Additive comprising tomato, broccoli, carrot, shitake mushrooms, cranberry, apple, and orange; a composition comprising dehydrated chicken, calcium carbonate, cesium chloride, and a Fruit- and Vegetable-Based Additive comprising cranberry, pomegranate, orange, grape, strawberry, and shitake mushrooms; a composition comprising dehydrated chicken, calcium carbonate, cesium chloride, and a Fruit- and Vegetable-Based Additive comprising spinach, broccoli, carrot, tomato, beet, and shitake mushrooms; a composition comprising dehydrated fish, calcium carbonate, cesium chloride, and a Fruit- and Vegetable-Based Additive comprising tomato, broccoli, carrot, shitake mushrooms, cranberry, apple, and orange; a composition comprising dehydrated fish, calcium carbonate, cesium chloride, and a Fruit- and Vegetable-Based Additive comprising cranberry, pomegranate, orange, grape, strawberry, and shitake mushrooms; a composition comprising dehydrated fish, calcium carbonate, cesium chloride, and a Fruit- and Vegetable-Based Additive comprising spinach, broccoli, carrot, tomato, beet, and shitake mushrooms; a composition comprising dehydrated chicken, calcium carbonate, cesium bicarbonate, and a Fruit- and Vegetable-Based Additive comprising tomato, broccoli, carrot, shitake mushrooms, cranberry, apple, and orange; a composition comprising dehydrated chicken, calcium carbonate, cesium bicarbonate, and a Fruit- and Vegetable-Based Additive comprising cranberry, pomegranate, orange, grape, strawberry, and shitake mushrooms; a composition comprising dehydrated chicken, calcium carbonate, cesium bicarbonate, and a Fruit- and Vegetable-Based Additive comprising spinach, broccoli, carrot, tomato, beet, and shitake mushrooms; a composition comprising dehydrated fish, calcium carbonate, cesium bicarbonate, and a Fruit- and Vegetable-Based Additive comprising tomato, broccoli, carrot, shitake mushrooms, cranberry, apple, and orange; a composition comprising dehydrated fish, calcium carbonate, cesium bicarbonate, and a Fruit- and Vegetable-Based Additive comprising cranberry, pomegranate, orange, grape, strawberry, and shitake mushrooms; a composition comprising dehydrated fish, calcium carbonate, cesium bicarbonate, and a Fruit- and Vegetable-Based Additive comprising spinach, broccoli, carrot, tomato, beet, and shitake mushrooms.

In another case, compositions according to the present invention consist essentially of Dehydrated Domestic Animal Food and a Phosphate Binder. In another case, compositions according to the present invention consist essentially of Dehydrated Domestic Animal Food and a Cesium Salt and/or a Rubidium Salt. In another case, compositions according to the present invention consist essentially of Dehydrated Domestic Animal Food and a Fruit-Based, Vegetable-Based, or a Fruit- and Vegetable-Based Additive.

In another case compositions according to the present invention consist essentially of Dehydrated Domestic Animal Food, a Phosphate Binder, and a Cesium Salt and/or a Rubidium Salt. In another case compositions according to the present invention consist essentially of Dehydrated Domestic Animal Food, a Phosphate Binder, and a Fruit-Based, Vegetable-Based, or a Fruit- and Vegetable-Based Additive. In another case, compositions according to the present invention consist essentially of Dehydrated Domestic Animal Food, a Cesium Salt and/or a Rubidium Salt, and a Fruit-Based, Vegetable-Based, or a Fruit- and Vegetable-Based Additive. In another case, compositions according to the present invention consist essentially of Dehydrated Domestic Animal Food, a Phosphate Binder, a Cesium Salt and/or a Rubidium Salt, and a Fruit-Based, Vegetable-Based, or a Fruit- and Vegetable-Based Additive.

In another case, compositions according to the present invention consist of Dehydrated Domestic Animal Food and a Phosphate Binder. In another case, compositions according to the present invention consist of Dehydrated Domestic Animal Food and a Cesium Salt and/or a Rubidium Salt. In another case, compositions according to the present invention consist of Dehydrated Domestic Animal Food and a Fruit-Based, Vegetable-Based, or a Fruit- and Vegetable-Based Additive.

In another case compositions according to the present invention consist of Dehydrated Domestic Animal Food, a Phosphate Binder, and a Cesium Salt and/or a Rubidium Salt. In another case compositions according to the present invention consist of Dehydrated Domestic Animal Food, a Phosphate Binder, and a Fruit-Based, Vegetable-Based, or a Fruit- and Vegetable-Based Additive. In another case, compositions according to the present invention consist of Dehydrated Domestic Animal Food, a Cesium Salt and/or a Rubidium Salt, and a Fruit-Based, Vegetable-Based, or a Fruit- and Vegetable-Based Additive. In another case, compositions according to the present invention consist of Dehydrated Domestic Animal Food, a Phosphate Binder, a Cesium Salt and/or a Rubidium Salt, and a Fruit-Based, Vegetable-Based, or a Fruit- and Vegetable-Based Additive.

The compositions of the present invention are used to treat chronic renal failure in domestic animals or symptoms related to chronic renal failure. In one case, a composition is provided to the domestic animal for ingestion by the animal. In another case, a composition is added to wet or dry food typically eaten by the animal.

Where one or more phosphate binders are in the composition used to treat the domestic animal, the amount of included phosphate binders is typically adjusted such that the phosphorus concentration in the animal's blood is less than 6.0 mg/dl. In certain cases, the adjustment provides a phosphorus concentration of less than 5.0 mg/dl or 4.5 mg/dl.

Where one or more cesium and/or rubidium salts are in the composition used to treat the domestic animal, the amount of salt included typically ranges from one milligram to one gram. The included amount is suitably varied to obtain a desired level of symptom reduction.

Where Fruit-, Vegetable- or Fruit- and Vegetable-Based additives are in the composition used to treat the domestic animal, the amount of additive included typically ranges from one milligram to ten grams. The included amount is suitably varied to obtain a desired level of symptom reduction.

Where Dehydrated Domestic Animal Food is in the composition to treat the domestic animal, the amount of dehydrated food included typically ranges from 100 milligrams to one kilogram. The included amount is suitably varied to obtain a desired level of symptom reduction.

1. A composition for the treatment of chronic renal failure in a domestic animal or symptoms related to chronic renal failure, wherein the composition comprises a Fruit- and Vegetable-Based Additive.

2. The composition according to claim 1, wherein the Fruit- and Vegetable-Based Additive is a concentrate, and wherein the concentrate comprises one or more fruits and vegetables from a list consisting of: tomato, cranberry, apple, orange, pomegranate, grape, strawberry, broccoli, carrot, shitake mushrooms, spinach, beet.

3. The composition according to claim 1, wherein the Fruit- and Vegetable-Based Additive is a concentrate, and wherein the concentrate comprises one or more fruits and vegetables from a list consisting of: tomato, broccoli, carrot, shitake mushrooms, cranberry, apple, and orange.

4. The composition according to claim 1, wherein the Fruit- and Vegetable-Based Additive is a concentrate, and wherein the concentrate comprises one or more fruits and vegetables from a list consisting of: cranberry, pomegranate, orange, grape, strawberry, and shitake mushrooms.

5. The composition according to claim 1, wherein the Fruit- and Vegetable-Based Additive is a concentrate, and wherein the concentrate comprises one or more fruits and vegetables from a list consisting of: spinach, broccoli, carrot, tomato, beet, and shitake mushrooms.

6. The composition according to claim 1, wherein the composition further comprises a phosphate binder.

7. The composition according to claim 1, wherein the composition further comprises a cesium or rubidium salt.

8. The composition according to claim 6, wherein the composition further comprises a cesium or rubidium salt.

9. The composition according to claim 8, wherein the phosphate binder is chitosan or calcium carbonate.

10. The composition according to claim 9, wherein the composition comprises a cesium salt, and wherein the cesium salt is cesium chloride or cesium bicarbonate.
